(19) United States
(12) Patent Application Publication
(10) Pub. No.: US 2023/0165801 A1
(43) Pub. Date: Jun. 1, 2023

(54) COMPOSITION FOR COATING TABLET

(71) Applicant: AMOREPACIFIC CORPORATION, Seoul (KR)

(72) Inventors: Kwanghyun SHIN, Yongin-si (KR); Wonkyung CHO, Yongin-si (KR); Minjung KWON, Yongin-si (KR); Wanki KIM, Yongin-si (KR); Chan Woong PARK, Yongin-si (KR)

(73) Assignee: AMOREPACIFIC CORPORATION, Seoul (KR)

(21) Appl. No.: 18/059,008

(22) Filed: Nov. 28, 2022

(30) Foreign Application Priority Data
Nov. 30, 2021 (KR) 10-2021-0168758

(51) Int. Cl. A61K 9/28; A61K 9/20; A61K 36/82

(52) U.S. Cl. CPC A61K 9/286; A61K 9/2866; A61K 9/288; A61K 9/2013; A61K 36/82; A61K 9/2893

(57) ABSTRACT

According to one aspect, the disclosure provides a composition for coating a tablet, including an alginate, a natural oil, a modified starch, and a cellulose derivative. According to another aspect, the disclosure provides a method for enteric coating of a tablet, including the step of coating a tablet with a composition including an alginate, a natural oil, a lecithin, and a cellulose derivative. The composition for coating a tablet according to an embodiment of the disclosure is safe as a naturally-derived ingredient and has excellent ability of coating because it can effectively coat even the edge of the tablet. In addition, the composition for coating a tablet according to an embodiment of the disclosure can implement an enteric coating, and thus has excellent heartburn suppression effect.

### CROSS REFERENCE TO RELATED APPLICATION

The present application claims priority to Korean Patent Application No. 10-2021-0168758, filed Nov. 30, 2021, the entire contents of which are hereby incorporated by reference.

### BACKGROUND OF THE INVENTION

### Field of the Invention

The present specification relates to a composition for coating a tablet.

### Description of the Related Art

Tablet products containing green tea extract and the like cause heartburn by stimulating the stomach when disintegrating and dissolving in stomach. In order to prevent such heartburn, tablets are usually enteric-coated.

Conventional coating materials for enteric coating of tablets are not preferred by consumers because they are synthetic additives. Also, since the coating is performed by spraying using an organic solvent, there are problems of explosion or fire hazard due to the organic solvent, environmental pollution due to release into the atmosphere, and toxicity due to residual organic solvent.

In order to solve this problem, an enteric coating material that can be coated using water has been developed, but there is a limit to the application in tablet formulations due to low ability of coating at the edge of the tablet.

Accordingly, there is a need to develop a safe, natural-derived coating composition capable of enteric coating for tablets.

### SUMMARY OF THE INVENTION

In one aspect, the present disclosure is to provide a composition for coating a tablet having excellent ability of coating.

In one aspect, the present disclosure is to provide a composition for coating a tablet capable of enteric coating and having an excellent heartburn suppression effect.

In one aspect, the disclosure provides a composition for coating a tablet, including an alginate, a natural oil, a modified starch, and a cellulose derivative.

As an exemplary embodiment, a weight ratio of the alginate and the natural oil may be 0.5 to 10:1.

As an exemplary embodiment, a weight ratio of the cellulose derivative and the natural oil may be 0.1 to 2:1.

As an exemplary embodiment, the alginate may include one or more selected from the group consisting of a sodium alginate, a potassium alginate, and an ammonium alginate.

As an exemplary embodiment, the natural oil may be a vegetable oil.

As an exemplary embodiment, the vegetable oil may include one or more selected from the group consisting of an avocado oil, a coconut oil, a lemon oil, an olive oil, a grape seed oil, a castor oil, a soybean oil, and a corn oil.

As an exemplary embodiment, the cellulose derivative may be a cellulose derivative substituted with one or more selected from the group consisting of a hydroxyl group, an alkyl group, and a hydroxyalkyl group, wherein the alkyl may be C1-C10 alkyl.

As an exemplary embodiment, the cellulose derivative may include one or more selected from the group consisting of a hydroxypropyl cellulose, a hydroxypropyl methyl cellulose, a methyl cellulose, and a hydroxyethyl cellulose.

As an exemplary embodiment, the modified starch may include one or more selected from the group consisting of an acid modified starch, an oxidized starch, a cross-linked starch, and a pregelatinized starch.

As an exemplary embodiment, a weight ratio of the modified starch and the natural oil may be 0.5 to 5:1.

As an exemplary embodiment, the composition may further include a lecithin.

As an exemplary embodiment, a weight ratio of the lecithin and the natural oil may be 0.01 to 0.2:1.

As an exemplary embodiment, the tablet may include a green tea extract.

As an exemplary embodiment, the composition for coating a tablet may be coated in a coating amount of 3 to 10% by weight based on a total weight of the tablet.

As an exemplary embodiment, the coating may be an enteric coating.

In one aspect, the composition for coating a tablet according to the present disclosure has excellent ability of coating, and can effectively coat a tablet formulation having an edge.

In one aspect, the composition for coating a table according to the present disclosure can be enteric coating.

In one aspect, the composition for coating a table according to the present disclosure has excellent enteric properties, thereby suppressing or preventing heartburn due to disintegration or dissolution of the tablet in the stomach.

### BRIEF DESCRIPTION OF THE DRAWINGS

FIG. 1 is a graph showing the measurement results of disintegration time of Example 1 according to an aspect of the present disclosure.

FIG. 2 is a graph showing the measurement results of disintegration time of Example 2 according to an aspect of the present disclosure.

### DETAILED DESCRIPTION OF THE INVENTION

Hereinafter, the present disclosure will be described in detail.

In one aspect, the present disclosure provides a composition for coating a tablet including an alginate, a natural oil, a modified starch, and a cellulose derivative.

In one embodiment, the content of the alginate may be 40 to 60% by weight based on the weight of the composition. If the content of the alginate is less than 40% by weight, the enteric properties may be reduced, and if the content of the alginate is more than 60% by weight, the ability of coating may be reduced. Specifically, the content of the alginate may be 40% by weight or more, 40.5% by weight or more, 41% by weight or more, 41.5% by weight or more, 42% by weight or more, 42.5% by weight or more, 43% by weight or more, 43.5% by weight or more, 44% by weight or more, 44.5% by weight or more, 45% by weight or more, 45.5% by weight or more, 46% by weight or more, 46.5% by weight or more, 47% by weight or more, 47.5% by weight or more, 48% by weight or more, 48.5% by weight or more, or 49% by weight or more, based on the weight of the composition, while the content of the alginate may be 60% by weight or less, 59.5% by weight or less, 59% by weight or less, 58.5% by weight or less, 58% by weight or less, 57.5% by weight or less, 57% by weight or less, 56.5% by weight or less, 56% by weight or less, 55.5% by weight or less, 55% by weight or less, 54.5% by weight or less, 54% by weight or less, 53.5% by weight or less, 53% by weight or less, 52.5% by weight or less, 52% by weight or less, 51.5% by weight or less, or 51% by weight or less, based on the weight of the composition.

In one embodiment, the alginate may include at least one selected from the group consisting of a sodium alginate, a potassium alginate, and an ammonium alginate.

In one embodiment, the weight ratio of the alginate and the natural oil may be 0.5 to 10:1. When the weight ratio of the alginate and the natural oil is out of the above range, the enteric properties of the coating may be deteriorated. Specifically, the weight ratio of the alginate and the natural oil may be 0.5 to 10:1, 1 to 10:1, 1 to 7:1, 2 to 7:1, 2 to 4.5:1, or 2 to 4:1.

In one embodiment, the content of the natural oil may be 10 to 20% by weight based on the weight of the composition. If the content of the natural oil is out of the above range, the ability of coating may be reduced. Specifically, the content of the natural oil may be 10% by weight or more, 10.5% by weight or more, 11% by weight or more, 11.5% by weight or more, 12% by weight or more, 12.5% by weight or more, or 13% by weight or more, based on the weight of the composition, while the content of the natural oil may be 20% by weight or less, 19.5% by weight or less, 19% by weight or less, 18.5% by weight or less, 18% by weight or less, 17.5% by weight or less, 17% by weight or less, 16.5% by weight or less, 16% by weight or less, 15.5% by weight or less, or 15% by weight or less, based on the weight of the composition.

In one embodiment, the natural oil may be a vegetable oil. For example, the vegetable oil may include at least one selected from the group consisting of an avocado oil, a coconut oil, a lemon oil, an olive oil, a grapeseed oil, a castor oil, a soybean oil, and a corn oil.

In one embodiment, the content of the cellulose derivative may be 1 to 10% by weight based on the weight of the composition. If the content of the cellulose derivative is out of the above range, the ability of coating may be reduced. Specifically, the content of the cellulose derivative may be 1% by weight or more, 1.5% by weight or more, 2% by weight or more, 2.5% by weight or more, 3% by weight or more, 3.5% by weight or more, or 4% by weight, based on the weight of the composition, while the content of the cellulose derivative may be 10% by weight or less, 9.5% by weight or less, 9% by weight or less, 8.5% by weight or less, 8% by weight or less, 7.5% by weight or less, 7% by weight or less, 6.5% by weight or less, or 6% by weight or less, based on the weight of the composition.

In one embodiment, the weight ratio of the cellulose derivative and the natural oil may be 0.1 to 2:1. If the weight ratio of the cellulose derivative and the natural oil is out of the above range, the enteric properties of the coating may be deteriorated. Specifically, the weight ratio of the cellulose derivative and the natural oil may be 0.1 to 2:1, 0.1 to 1.5:1, 0.2 to 1.5:1, or 0.2 to 1:1.

In one embodiment, the cellulose derivative is a cellulose derivative substituted with one or more selected from the group consisting of a hydroxyl group, an alkyl group, and a hydroxyalkyl group, and the alkyl may be C1-C10 alkyl.

In one embodiment, the cellulose derivative may include one or more selected from the group consisting of a hydroxypropyl cellulose, a hydroxypropyl methyl cellulose, a methyl cellulose, and a hydroxyethyl cellulose.

In one embodiment, the content of the modified starch may be 20 to 40% by weight based on the weight of the composition. If the content of the modified starch is out of the above range, the ability of coating may be reduced. Specifically, the content of the modified starch may be 20% by weight or more, 20.5% by weight or more, 21% by weight or more, 21.5% by weight or more, 22% by weight or more, 22.5% by weight or more, 23% by weight or more, 23.5% by weight or more, 24% by weight or more, 24.5% by weight or more, 25% by weight or more, 25.5% by weight or more, 26% by weight or more, 26.5% by weight or more, 27% by weight or more, 27.5% by weight or more, or 28% by weight or more, based on the weight of the composition, while the content of the modified starch may be 40% by weight or less, 39.5% by weight or less, 39% by weight or less, 38.5% by weight or less, 38% by weight or less, 37.5% by weight or less, 37% by weight or less, 36.5% by weight or less, 36% by weight or less, 35.5% by weight or less weight % or less, 35% by weight or less, 34.5% by weight or less, 34% by weight or less, 33.5% by weight or less, 33% by weight or less, 32.5% by weight or less, or 32% by weight or less, based on the weight of the composition.

In one embodiment, the weight ratio of the modified starch and the natural oil may be 0.5 to 5:1. If the weight ratio of the modified starch and the natural oil is out of the above range, the ability of coating may be reduced. Specifically, the weight ratio of the modified starch and the natural oil may be 0.5 to 5:1, 2 to 5:1, 1 to 3.5:1, or 1.5 to 2.5:1.

In one embodiment, the modified starch refers to the starch obtained by chemically, physically or enzymatically treating natural starch. For example, the modified starch may include one or more selected from the group consisting of acid modified starch, oxidized starch, cross-linked starch, and pregelatinized starch.

In one embodiment, the composition for coating a tablet according to the present disclosure may further include a lecithin.

In one embodiment, the content of the lecithin may be 0.1 to 10% by weight based on the weight of the composition. If the content of lecithin is out of the above range, formulation stability may be reduced. Specifically, the content of the lecithin may be 0.1% by weight or more, 0.2% by weight or more, 0.3% by weight or more, 0.4% by weight or more, 0.5% by weight or more, 0.6% by weight or more, 0.7% by weight or more, 0.8% by weight or more, 0.9% by weight or more, 1% by weight or more, 1.1% by weight or more, 1.2% by weight or more, 1.3% by weight or more, 1.4% by weight or more, 1.5% by weight or more, 1.6% by weight or more, 1.7% by weight or more, 1.8% by weight or more, 1.9% by weight or more, or 2% by weight or more, based on the weight of the composition, while the content of the lecithin may be 10% by weight or less, 9.9% by weight or less, 9.8% by weight or less, 9.7% by weight or less, 9.6% by weight or less, 9.5% by weight or less, 9.4% by weight or less, 9.3% by weight or less, 9.2% by weight or less, 9.1% by weight or less, 9% by weight or less, 8.9% by weight or less, 8.8% by weight or less, 8.7% by weight or less, 8.6% by weight or less, 8.5% by weight or less, 8.4% by weight or less, 8.3% by weight or less, 8.2% by weight or less, 8.1% by weight or less, or 8% by weight or less.

In one embodiment, the weight ratio of the lecithin and the natural oil may be 0.01 to 0.2:1. If the weight ratio of the lecithin and the natural oil is out of the above range, formulation stability may be deteriorated. Specifically, the weight ratio of the lecithin and the natural oil may be 0.01 to 0.2:1, 0.01 to 0.15:1, 0.03 to 0.15:1, or 0.03 to 1:1.

In one embodiment, the tablet is not particularly limited, but may include green tea extract as an active ingredient. The green tea extract may include an extract extracted from tea (Camellia sinensis), an evergreen tree belonging to the Tea plant family, or an extract extracted from tea leaves, etc. inoculated and fermented with Bacillus subtilis spp. regardless of an extraction method, an extraction solvent, an extracted component, or a form of the extract, and a fraction obtained by fractionation of the extract with a specific solvent. The tea includes one or more selected from the group consisting of tea leaves, flowers, stems, fruits, roots, stems, and heartwood of roots, and may preferably be leaves. In addition, the form of the extract may be preferably in the form of a powder. The extraction or fractionation may be performed using water, an organic solvent, or a mixed solvent thereof. The organic solvent may use alcohol, isopropanol, acetone, hexane, ethyl acetate, carbon dioxide, or a mixed solvent of two or more thereof, but is not limited thereto. It can be extracted or fractionated by heating or at room temperature under conditions in which the active ingredient of green tea is not destroyed or minimized. The alcohol may be a C₁ to C5 lower alcohol. The number or method of extraction or fractionation is not particularly limited, and for example, methods such as cold extraction, ultrasonic extraction, reflux cooling extraction, hot water extraction, etc. may be used. Preferably, the green tea extract of the present disclosure may be obtained by extracting, fractioning, or filtering the active ingredient with cooling or heating, and concentrating the filtrate under reduced pressure.

In one embodiment, the coating may be an enteric coating.

In the present specification, the enteric coating is a coating that prevents the tablet from being dissolved or released by gastric acid before reaching the intestine, and refers to a coating that can be expected to disintegrate or elute the tablet in the small intestine. Such an enteric coating prevents the tablet or the like from being decomposed in gastric acid and irritating the stomach wall, or allows the tablet or the like to be absorbed more efficiently in the intestinal tract.

In one embodiment, the composition for coating a tablet may be coated in a coating amount of 3 to 10% by weight based on the total weight of the tablet. Specifically, the composition for coating a tablet may be coated in a coating amount of 3% by weight or more, 3.1% by weight or more, 3.2% by weight or more, 3.3% by weight or more, 3.4% by weight or more, 3.5% by weight or more, 3.6% by weight or more, 3.7% by weight or more, 3.8% by weight or more, 3.9% by weight or more, 4% by weight or more, 4.1% by weight or more, 4.2% by weight or more, 4.3% by weight or more, 4.4% by weight or more, or 4.5% by weight or more, based on the total weight of the tablet, while the composition for coating a tablet may be coated in a coating amount of 10% by weight or less, 9.9% by weight or less, 9.8% by weight or less, 9.7% by weight or less, 9.6% by weight or less, 9.5% by weight or less, 9.4% by weight or less, 9.3% by weight or less, 9.2% by weight or less, 9.1% by weight or less, 9% by weight or less, 8.9% by weight or less, 8.8% by weight or less, 8.7% by weight or less, 8.6% by weight or less, or 8.5% by weight or less, based on the total weight of the tablet.

In one aspect, the present disclosure provides a method for enteric coating of a tablet, including the step of coating a tablet with a composition including an alginate, a natural oil, a lecithin, and a cellulose derivative. In one aspect, the coating of the method may be performed with the composition and coating method described in the present disclosure.

In one aspect, the present disclosure provides the use of the composition including an alginate, a natural oil, a lecithin, and a cellulose derivative for tablet coating.

In one aspect, the present disclosure provides the use of the alginate, natural oil, lecithin, and cellulose derivative for preparing a composition for coating a tablet.

Hereinafter, the present invention will be described in more detail through examples. These examples are only for illustrating the present invention, and it will be apparent to those of ordinary skill in the art that the scope of the present invention is not to be construed as being limited by these examples.

### [Preparation Example 1] Preparation of a Tablet

680 g of green tea extract (Borak, Korea) and 119.2 g of lactose were put in a fluidized bed granulator (GPCG-1, Glatt, Germany), and 0.8 g of guar gum was dissolved in 160 g of purified water. Granules were prepared under the process conditions of a supply air temperature of 70° C., an air volume of 50 cmh, and a spray rate of 9 g/min. 495 g of green tea extract granules prepared in this way were mixed with 5 g of magnesium stearate for about 5 minutes, and then a tablet containing the green tea extract was prepared using a single tablet press (XP-1, Korsch, Germany) to obtain a hardness of 8-12 kp.

### EXAMPLE

According to the composition of Table 1 below, the coating compositions of Examples 1 to 3 were prepared.

TABLE 1

| (wt %) | Example 1 | Example 2 | Example 3 |
|---|---|---|---|
| Sodium alginate | 47.9 | 47.9 | 47.9 |
| Starch acetate(Eudragard ® | 31.9 | 31.9 | 31.9 |
| natural, Evonik) | | | |
| Hydroxypropyl cellulose | 4.2 | 4.2 | 4.2 |
| (Klucel ™, Ashland) | | | |
| Lecithin | 0.8 | 0.8 | |
| Coconut oil | | 15.2 | 16 |
| Avocado oil | 15.2 | | |
| Total | 100 | 100 | 100 |

### Comparative Example

According to the composition of Table 2 below, the coating compositions of Comparative Examples 1 to 4 were prepared and used.

TABLE 2

| | Comparative | Comparative | Comparative | Comparative |
|---|---|---|---|---|
| (wt %) | Example 1 | Example 2 | Example 3 | Example 4 |
| Sodium alginate | 50.4 | 47.9 | 47.9 | 47.9 |
| Starch acetate (Eudragard ® | 33.6 | 31.9 | 31.9 | 31.9 |
| natural, Evonik) | | | | |
| Hydroxypropylmethyl cellulose | | 4.2 | 4.2 | 4.2 |
| (Methocel ™, IFF) | | | | |
| Glycerin | 16 | 16 | 16 | 15.2 |
| Lecithin | | | | 0.8 |
| Total | 100 | 100 | 100 | 100 |

### [Experimental Example 1] Disintegration Test

The coating compositions of Examples 1 to 3 and Comparative Examples 1 to 4 were mixed with purified water, and 500 g of the tablets of Preparation Example 1 were coated with the coating composition under the coating conditions of Table 3 below. The coating amount was 7% by weight of the tablet.

TABLE 3

| | Condition | |
|---|---|---|
| Coater | Solidlab 1 (Syntegon, Germany) | |
| Supply air temperature | 52° C. | |
| Exhaust temperature | 35 to 38° C. | |
| Air volume | 60 | m³/hr |
| Injection pressure | 0.8 | bar |
| Injection speed | 5 | g/min |
| Fan speed | 22 | rpm |

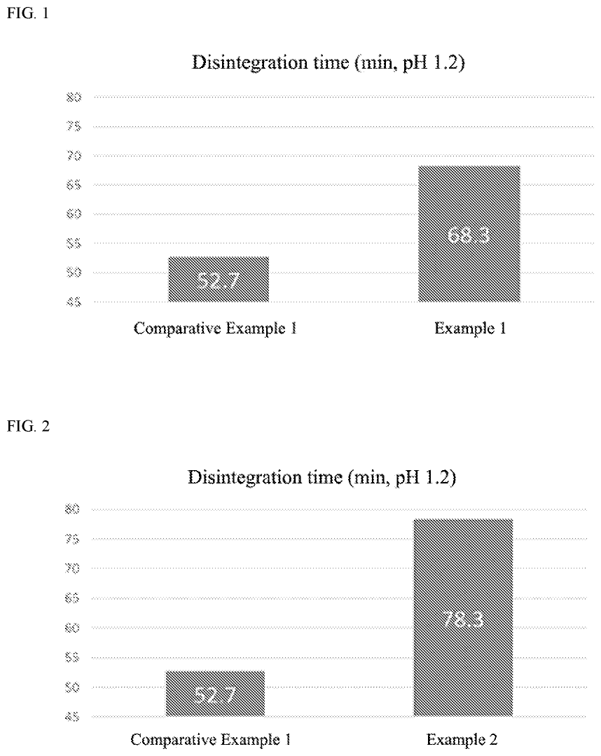

The tablets thus coated were subjected to a disintegration test with the solution 1 (pH 1.2) in the disintegration test method described in the Korean Pharmacopeia, and the average disintegration time is shown in Table 4 and FIGS. 1 to 2 below.

TABLE 4

| | Comp. | Comp. | Comp. | Comp. | | | |
|---|---|---|---|---|---|---|---|
| | Ex. 1 | Ex. 2 | Ex. 3 | Ex. 4 | Ex. 1 | Ex. 2 | Ex. 3 |
| Average | 52.7 | 53.1 | 55.8 | 55.4 | 78.3 | 70.7 | 68.9 |
| disintegration | | | | | | | |
| time (min) | | | | | | | |

As can be seen from the results of Table 4, it was confirmed that the average disintegration time of Examples 1 to 3 was significantly longer than that of Comparative Examples 1 to 4. As the disintegration time was longer, less ingredients of the tablet that could cause heartburn leaked, so it was confirmed that the heartburn suppression effect of Examples 1 to 3 was greater. In particular, the enteric standard for dietary supplements in US is not disintegrating for 60 minutes in artificial gastric juice (representing a pH of 1.2). Therefore, Examples 1 to 3 satisfied the enteric standard, but Comparative Examples 1 to 4 did not satisfy the enteric standard.

What is claimed is:

1. A composition for coating a tablet, comprising an alginate, a natural oil, a modified starch, and a cellulose derivative.

2. The composition of claim 1, wherein a weight ratio of the alginate and the natural oil is 0.5 to 10:1,
a weight ratio of the cellulose derivative and the natural oil is 0.1 to 2:1, or
a weight ratio of the modified starch and the natural oil is 0.5 to 5:1.

3. The composition of claim 1, wherein the alginate includes one or more selected from the group consisting of a sodium alginate, a potassium alginate, and an ammonium alginate,
the natural oil is a vegetable oil, or
the modified starch includes one or more selected from the group consisting of an acid modified starch, an oxidized starch, a cross-linked starch, and a pregelatinized starch.

4. The composition of claim 3, wherein the vegetable oil includes one or more selected from the group consisting of an avocado oil, a coconut oil, a lemon oil, an olive oil, a grape seed oil, a castor oil, a soybean oil, and a corn oil.

5. The composition of claim 1, wherein the cellulose derivative is a cellulose derivative substituted with one or more selected from the group consisting of a hydroxyl group, an alkyl group, and a hydroxyalkyl group, wherein the alkyl is C1-C10 alkyl, or
the cellulose derivative includes one or more selected from the group consisting of a hydroxypropyl cellulose, a hydroxypropyl methyl cellulose, a methyl cellulose, and a hydroxyethyl cellulose.

6. The composition of claim 1, further comprising a lecithin.

7. The composition of claim 6, wherein a weight ratio of the lecithin and the natural oil is 0.01 to 0.2:1.

8. The composition of claim 1, wherein the tablet includes a green tea extract.

9. The composition of claim 1, wherein the composition for coating a tablet is coated in a coating amount of 3 to 10% by weight based on a total weight of the tablet.

10. The composition of claim 1, wherein the coating is an enteric coating.

11. A method for enteric coating of a tablet, comprising the step of coating a tablet with a composition comprising an alginate, a natural oil, a lecithin, and a cellulose derivative.

12. The method of claim 11, wherein a weight ratio of the alginate and the natural oil is 0.5 to 10:1,
a weight ratio of the cellulose derivative and the natural oil is 0.1 to 2:1, or
a weight ratio of the modified starch and the natural oil is 0.5 to 5:1.

13. The method of claim 11, wherein the alginate includes one or more selected from the group consisting of a sodium alginate, a potassium alginate, and an ammonium alginate,
the natural oil is a vegetable oil, or
the modified starch includes one or more selected from the group consisting of an acid modified starch, an oxidized starch, a cross-linked starch, and a pregelatinized starch.

14. The method of claim 13, wherein the vegetable oil includes one or more selected from the group consisting of an avocado oil, a coconut oil, a lemon oil, an olive oil, a grape seed oil, a castor oil, a soybean oil, and a corn oil.

15. The method of claim 11, wherein the cellulose derivative is a cellulose derivative substituted with one or more selected from the group consisting of a hydroxyl group, an alkyl group, and a hydroxyalkyl group, wherein the alkyl is C1-C10 alkyl, or
the cellulose derivative includes one or more selected from the group consisting of a hydroxypropyl cellulose, a hydroxypropyl methyl cellulose, a methyl cellulose, and a hydroxyethyl cellulose.

16. The method of claim 11, wherein the composition further comprises a lecithin.

17. The method of claim 16, wherein a weight ratio of the lecithin and the natural oil is 0.01 to 0.2:1.

18. The method of claim 11, wherein the tablet includes a green tea extract.

19. The method of claim 11, wherein in the step of coating the tablet, the composition is coated in a coating amount of 3 to 10% by weight based on a total weight of the tablet.

20. The method of claim 11, wherein the coating is an enteric coating.
